(19) United States
(12) Patent Application Publication
(10) Pub. No.: US 2008/0006279 A1
(43) Pub. Date: Jan. 10, 2008

(54) Drapes

(75) Inventors: Andrew Bodenham, Thorner (GB); Timothy Bateman, Dymchurch (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/819,269

(22) Filed: Jun. 26, 2007

(30) Foreign Application Priority Data
Jun. 29, 2006 (GB) 0612884.7

(51) Int. Cl. A61B 19/08

(52) U.S. Cl. 128/853

(57) ABSTRACT

A surgical drape for use in tracheostomy procedures has a main body region and a transparent region arranged to extend over the head of the patient. An incise aperture is formed in the body region close to the transparent head region. An adhesive border around the patient side of the aperture enables the drape to be secured to the skin around the tracheostomy. A perforated tear line extends laterally from the aperture to the edge of the drape to enable the drape to be torn easily so that it can be removed from around any tubing projecting through the aperture.

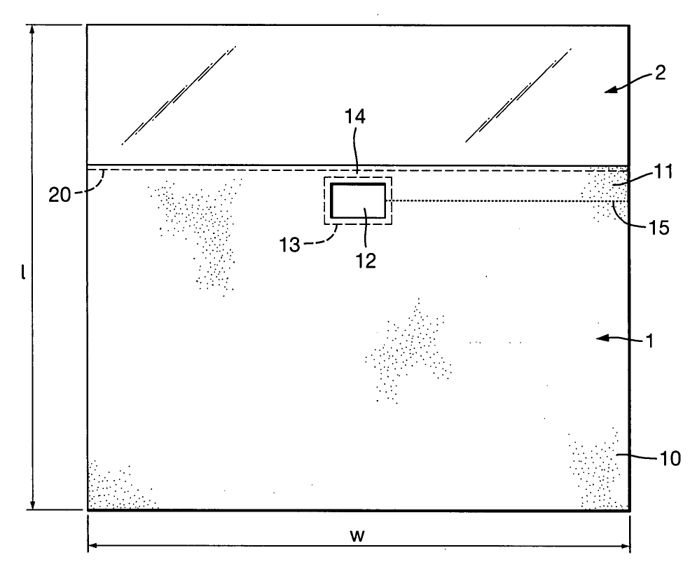

### BACKGROUND OF THE INVENTION

This invention relates to drapes.

The invention is more particularly concerned with drapes during surgical procedures on the neck, such as tracheostomy procedures.

Drapes are commonly used during surgical procedures to cover regions around the surgical site so as to protect the site from contamination. The drapes take many different forms. U.S. Pat. No. 4,384,573 describes a drape covering the entire body and having windows in the region of the abdomen and the face. U.S. Pat. No. 5,592,952 describes a drape with an aperture through which a limb of the patient is extended. U.S. Pat. No. 6,843,252 describes a drape held above the patient by a maintenance cover. U.S. Pat. No. 3,349,765 describes a rolled drape. U.S. Pat. No. 3,260,260 describes a drape with an adhesive layer. U.S. Pat. No. 6,742,522 and U.S. Pat. No. 5,803,086 describe incise drapes through which a cut can be made. U.S. Pat. No. 5,341,821 describes a drape that hangs down the sides of an operating table. U.S. Pat. No. 6,694,981 describes a drape with a central transparent region.

Presently available drapes are not ideally suited to tracheostomy or other procedures in the region of the neck. It is common practice during such procedures to use up to three different drapes laid one over the other in order to cover the regions of the patient that need to be covered.

### BRIEF SUMMARY OF THE PRESENT INVENTION

It is an object of the present invention to provide an alternative drape.

According to the present invention there is provided a surgical drape for use during a surgical procedure in the region of the neck, the drape being of generally rectangular shape and of a size to extend from the top of the head to the groin region, the drape having a first, transparent region extending across the width of the drape at the head end to enable the surgeon to view the head of the patient through the first region, a second, opaque region extending over the remainder of the drape, an incise window formed in the second opaque region adjacent the first region and located to be positioned over the neck of the patient, and an adhesive material on the patient side of the drape in the region of the incise window to enable the drape to be adhered to the patient's skin in the region of the incise window.

The incise window is preferably an open aperture. The adhesive material may be a strip extending as a border around the incise window. The drape may include a tear line extending from the incise window to an edge of the drape. The tear line preferably extends laterally across the second region and may be perforated. The second region may be of a woven, water-repellent material and the first region may be of polyethylene. The drape is preferably substantially 140 cm long and 160 cm wide. The first region is preferably substantially 40 cm long.

A tracheostomy drape according to the present invention will now be described, by way of example, with reference to the accompanying drawing.

### BRIEF DESCRIPTION OF THE DRAWING

The accompanying drawing is a plan view of the drape.

### DETAILED DESCRIPTION OF THE PREFERRED EMBODIMENT

The drape is rectangular with a length l of 140 cm and a width w of 160 cm. The drape is divided into a body region 1 and a head region 2.

The body region 1 is formed of a soft, spun-woven, flexible, drapeable sheet of an opaque, water-repellent single ply material. The body region 1 extends for 100 cm with its lower end 10 located, in use, in the region of the groin of the patient, and its upper end 11 located in the region of the chin. The body region 1 extends across the entire width w of the drape, this width being sufficient to cover the upper part of the body and hang down a short distance at opposite sides of the patient. The body region 1 is interrupted by an incise window 12 in the form of an open aperture cut through the material of the region and located a few centimetres from the edge of the upper end 11. The incise window 12 is located centrally across the width of the drape and is rectangular in shape being 10 cm wide and 15 cm long. The window could be of other shapes, such as square or oval. The location and dimensions of the incise window 12 are such that it is lies above the surface of the neck and enables ready access to the neck through the window. The underside of the drape, facing the patient, has a strip of a skin-compatible adhesive 13 extending as a border around the window 12. The body region 1 also has a straight perforation, tear line 15 extending laterally from the window 12 to the right-hand edge of the region. Instead of being perforated, the tear line could be provided in other ways, such as by a region of tearable material.

The head region 2 differs from the body region 1 in that it is formed of a clear, transparent, waterproof, flexible polyethylene material. The head region 2 is shorter than the body region 1, being 40 cm long, which is sufficient to cover the head. The lower edge 20 of the head region overlaps the patient side of the upper end 11 of the body region 1 and is securely bonded with it. It can be seen that the incise window 12 is separated from the clear head region 2 by an intervening strip 14 of the opaque body region material.

The drape is normally be supplied folded or rolled, with a strip of a release sheet (not shown) covering the adhesive 13. Alternatively, however, the adhesive could be protected by contact with the material of the body or head regions 1 or 2 itself.

In use, the drape is unfolded and placed over the torso of the patient with the head region 2 extending across the head and with the body region 1 extending down to the region of the groin, covering the arms, and abdomen of the patient. The incise window 12 is positioned over the neck and the user pushes the adhesive 13 into contact with the skin around the site of the planned tracheostomy procedure. This holds the drape secure in the region of the tracheostomy and provides clear access. The surgeon and anaesthetist can view the patient's face during the procedure to monitor for any changes. The drape provides effective protection in the region around the surgery without the need for multiple drapes. The drape can be readily removed even when a tube protrudes through the window 12 and is connected to ventilation tubing. The clinician tears along the perforation line 15 so that the drape can be removed without the need to disconnect the tube from the ventilation tubing.

Instead of the incise window being an open aperture, as described above, it could have a thin, flexible, transparent, polymer film with an adhesive surface extending across the aperture. The film is such that it can be adhered to the skin and cut through into the skin. This would obviate the need for a separate adhesive border around the incise aperture.

What we claim is:

1. A surgical drape for use during a surgical procedure in the region of the neck, the drape being of generally rectangular shape with a width and a length and of a size to extend from the top of a head to a groin region of a patient, wherein the drape comprises: a first region, said first region being transparent and extending across the width of the drape at a head end to enable a surgeon to view the head of the patient through said first region; a second region, said second region being opaque and extending over a remainder of the drape; an incise window, said incise window being formed in said second, opaque region adjacent said first region and located to be positioned over a neck of the patient; and an adhesive material, said adhesive material being disposed on a patient side of the drape in the region of said incise window to enable the drape to be adhered to the patient's skin in the region of said incise window.

2. A surgical drape according to claim 1, wherein said incise window is an open aperture.

3. A surgical drape according to claim 1, wherein said adhesive material is in a strip extending as a border around said incise window.

4. A surgical drape according to claim 1 including a tear line, said tear line extending from said incise window to an edge of the drape.

5. A surgical drape according to claim 4, wherein said tear line extends laterally across said second region.

6. A surgical drape according to claim 4, wherein said tear line is perforated.

7. A surgical drape according to claim 1, wherein said second region is of a woven, water-repellent material.

8. A surgical drape according to claim 1, wherein said first region is of polyethylene.

9. A surgical drape according to claim 1, wherein the drape is substantially 140 cm long and 160 cm wide.

10. A surgical drape according to claim 1, wherein said first region is substantially 40 cm long.

11. A tracheostomy drape of generally rectangular shape with a width and a length and with a size to extend from the top of a head to a groin region of a patient, wherein the drape comprises: a first region, said first region being of a transparent material and extending across the width of the drape at a head end to enable a surgeon to view the head of the patient through said first region; a second region, said second region being of a material different from that of said first region and extending over a remainder of the drape; an aperture, said aperture being formed in said second region adjacent said first region and located to be positioned over a neck of the patient; an adhesive border, said adhesive border extending around said aperture on a patient side of the drape to enable the drape to be adhered to the patient's skin around a tracheostomy; and a perforated line extending from said aperture across said second region to an edge of the drape.
